(19) United States
(12) Patent Application Publication
(10) Pub. No.: US 2024/0008548 A1
(43) Pub. Date: Jan. 11, 2024

(54) VAPORIZATION ASSEMBLY, VAPORIZER, AND ELECTRONIC VAPORIZATION DEVICE

(71) Applicant: Shenzhen Verdewell Technology Limited, Shenzhen (CN)

(72) Inventor: Jianguang ZHOU, Shenzhen (CN)

(21) Appl. No.: 18/342,053

(22) Filed: Jun. 27, 2023

(30) Foreign Application Priority Data
Jul. 6, 2022 (CN) 202221723101.8

(51) Int. Cl. A24F 40/485; A24F 40/42; A24F 40/10

(52) U.S. Cl. CPC A24F 40/485; A24F 40/42; A24F 40/10

(57) ABSTRACT

A vaporization assembly includes: a ferrule body having an inner cavity; a vaporization body adapted into the inner cavity and provided with a liquid inlet hole; and a blocking portion protruding from a cavity wall of the inner cavity and arranged to surround a periphery of the vaporization body, the blocking portion being provided with at least one liquid-through clearance. The ferrule body is movable in a preset direction relative to the vaporization body and has pre-loading and conduction positions. When the ferrule body is located at the pre-loading position, the blocking portion partitions the inner cavity into a liquid storage cavity and a mounting cavity in the preset direction, and the liquid inlet hole is located in the mounting cavity and isolated from the liquid storage cavity. Movement of the ferrule body from the pre-loading position to the conduction position reduces a volume of the mounting cavity.

### CROSS-REFERENCE TO PRIOR APPLICATION

Priority is claimed to Chinese Patent Application No. 202221723101.8, filed on Jul. 6, 2022, the entire disclosure of which is hereby incorporated by reference herein.

### FIELD

The utility model relates to the field of electronic vaporization technologies, and in particular, to a vaporization assembly, a vaporizer and an electronic vaporization device.

### BACKGROUND

During production of a vaporizer, it is required to first inject a liquid, and then mount a mouthpiece assembly. After liquid injection is completed and before the mouthpiece assembly is mounted, e-liquid inside the vaporizer may seep through a heating assembly inside the vaporizer under the effect of atmospheric pressure and gravity, and may continue to seep through the bottom via a heating assembly, which causes liquid seepage. Meanwhile, during the mounting of the mouthpiece assembly, e-liquid is more likely to seep and leak under the squeezing action of the mouthpiece assembly, which causes waste of e-liquid and affects the functional performance of the vaporizer.

### SUMMARY

In an embodiment, the present invention provides a vaporization assembly, comprising: a ferrule body having an inner cavity; a vaporization body adapted into the inner cavity and provided with a liquid inlet hole; and a blocking portion protruding from a cavity wall of the inner cavity and arranged to surround a periphery of the vaporization body, the blocking portion being provided with at least one liquid-through clearance, wherein the ferrule body is movable in a preset direction relative to the vaporization body and comprises a pre-loading position and a conduction position, wherein, when the ferrule body is located at the pre-loading position, the blocking portion partitions the inner cavity into a liquid storage cavity and a mounting cavity in the preset direction, and the liquid inlet hole is located in the mounting cavity and isolated from the liquid storage cavity, and wherein, during a movement of the ferrule body from the pre-loading position to the conduction position, a volume of the mounting cavity is reduced so as to squeeze liquid in the mounting cavity to flow back into the liquid storage cavity via the at least one liquid-through clearance.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic diagram of an overall structure of a vaporizer assembled without a mouthpiece assembly according to an embodiments of this application;

FIG. 2 is a sectional view of the vaporizer assembled without a mouthpiece assembly as shown in FIG. 1;

FIG. 3 is a schematic diagram of an overall structure of a ferrule body in the vaporizer according to an embodiment of this application;

FIG. 4 is a side view of the ferrule body as shown in FIG. 3;

FIG. 5 is a sectional view of the ferrule body as shown in FIG. 3;

FIG. 6 is a schematic diagram of an overall structure of a vaporization body in the vaporizer according to an embodiment of this application;

FIG. 7 is a sectional view of the vaporization body as shown in FIG. 6;

FIG. 8 is a sectional view of a vaporizer assembled with a mouthpiece assembly according to an embodiment of this application; and

FIG. 9 is a schematic diagram of an overall structure of the vaporizer according to an embodiment of this application.

### DETAILED DESCRIPTION

In an embodiment, the present invention provides a vaporization assembly, a vaporizer and an electronic vaporization device, so as to solve the problem of seepage and leakage of e-liquid.

In an embodiment, the present invention provides a vaporization assembly including:
a ferrule body having an inner cavity;
a vaporization body adapted into the inner cavity and provided with a liquid inlet hole; and
a blocking portion protruding from the cavity wall of the inner cavity and arranged to surround a periphery of the vaporization body, where the blocking portion is provided with a liquid-through clearance;
where the ferrule body is movable in a preset direction relative to the vaporization body, and includes a pre-loading position and a conduction position;
when the ferrule body is located at the pre-loading position, the blocking portion partitions the inner cavity into a liquid storage cavity and a mounting cavity in the preset direction, and the liquid inlet hole is located in the mounting cavity and isolated from the liquid storage cavity; and
when the ferrule body moves from the pre-loading position to the conduction position, the volume of the mounting cavity is reduced and the liquid in the mounting cavity is squeezed to flow back into the liquid storage cavity via the liquid-through clearance.

In some embodiments, the liquid-through clearance is arranged at one end of the blocking portion in contact with the vaporization body.

In some embodiments, at least two liquid-through clearances are provided and are uniformly arranged in the circumferential direction of the blocking portion.

In some embodiments, the area of the liquid-through clearance ranges from 0.1 square millimeter to 0.5 square millimeter. In some embodiments, the liquid-through clearance is configured to be a notch formed by recessing an edge of the blocking portion in contact with the vaporization body in a direction away from the vaporization body.

In some embodiments, the vaporization body includes a vaporization base, the surface of the vaporization base protrudes to form a limiting flange, and the ferrule body abuts against the limiting flange in a limiting manner when moving from the pre-loading position to the conduction position.

In some embodiments, the ferrule body moves between the pre-loading position and the conduction position relative to the vaporization body along its own axis.

According to a second aspect, this application provides a vaporizer including a vaporization assembly and a mouthpiece assembly, where the vaporization assembly is described above, and the mouthpiece assembly is movably adapted onto the ferrule body.

In some embodiments, the mouthpiece assembly is separated from the ferrule body when the ferrule body is located at the pre-loading position, and presses the ferrule body in the preset direction when assembled on the ferrule body to provide a downward pressure for the ferrule body to move from the pre-loading position to the conduction position.

According to a third aspect, this application provides an electronic vaporization device including a power supply assembly and the foregoing vaporizer, where the power supply assembly is electrically connected to the vaporizer.

Regarding the vaporization assembly, the vaporizer and the electronic vaporization device, when the ferrule body is located at the pre-loading position, the liquid inlet hole is located in the mounting cavity, and in this way, the liquid inlet hole and the liquid storage cavity are isolated by the blocking portion, thereby reducing the probability that the liquid in the liquid storage cavity enters the vaporization body via the liquid injection hole. In this way, a certain clearance is formed between the cavity wall of the mounting cavity and the liquid inlet hole, and the liquid in the liquid storage cavity may seep into the above clearance by other means, such as a joint clearance between structures. Therefore, when the ferrule body moves from the pre-loading position to the conduction position, the clearance between the cavity wall of the mounting cavity and the liquid inlet hole is filled by the vaporization body. During this process, the volume of the mounting cavity decreases continuously, and the liquid seeping into the mounting cavity can flow back into the liquid storage cavity via the liquid-through clearance on the blocking portion under the squeezing action. In this way, it can not only make fuller use of the liquid, but also prevent the situation that liquid seeping into the mounting cavity enters the vaporization body under the squeezing action and is inhaled by the human body.

Reference Numerals: 100. vaporizer; 10. vaporization assembly; 20. mouthpiece assembly; 11. ferrule body; 12. vaporization body; 13. blocking portion; 111. inner cavity; 121. liquid inlet hole; 122. vaporization base; 123. vaporization tube; 131. liquid-through clearance; 1111. liquid storage cavity; 1112. mounting cavity; 1221. limiting flange; 1231. liquid absorption assembly; 1232. heating assembly; and a. preset direction.

To make the foregoing objects, features and advantages of the utility model more apparent and comprehensible, a detailed description is made to the specific implementations of the utility model below with reference to the accompanying drawings. In the following description, many specific details are described for a thorough understanding of the utility model. However, the utility model may be implemented in many other manners different from those described herein. A person skilled in the art may make similar improvements without departing from the connotation of the utility model. Therefore, the utility model is not limited to the specific embodiments disclosed below.

It should be understood that, in the description of the utility model, the terms such as “central”, “longitudinal”, “transverse”, “length”, “width”, “thickness”, “upper”, “lower”, “front”, “back”, “left”, “right”, “vertical”, “horizontal”, “top”, “bottom”, “inner”, “outer”, “clockwise”, “anticlockwise”, “axial”, “radial” and “circumferential” are intended to indicate orientations shown in the drawings. It should be noted that these terms are merely intended to facilitate a simple description of the utility model, rather than to indicate or imply that the mentioned device or elements should have the specific orientation or be constructed and operated in the specific orientation. Therefore, these terms may not be construed as a limitation to the utility model.

In addition, terms “first” and “second” are used merely for the purpose of description, and shall not be construed as indicating or implying relative importance or implicitly indicating a quantity of indicated technical features. Therefore, a feature defined by “first” or “second” may explicitly indicate or implicitly include at least one of such features. In the description of the utility model, “multiple” means at least two, for example, two or three, unless otherwise clearly and specifically defined.

In the utility model, it should be noted that unless otherwise explicitly specified and defined, the terms “mount”, “connect”, “connection”, and “fix” should be understood in a broad sense. For example, a connection may be a fixed connection, a detachable connection, or an integral connection; or the connection may be a mechanical connection or an electrical connection; or the connection may be a direct connection, an indirect connection through an intermediary, or internal communication between two elements or interaction between two elements, unless otherwise defined explicitly. A person of ordinary skill in the art can understand specific meanings of the terms in the present utility model according to specific situations.

In the utility model, unless otherwise explicitly specified or defined, a first feature being “above” or “below” a second feature may refer to the first and second features being in direct contact or the first and second features being in indirect contact through an intermediary. In addition, the first feature being “above”, “over”, or “on” the second feature may refer to the first feature being directly or obliquely above the second feature, or may merely indicate that the horizontal position of the first feature is greater than that of the second feature. The first feature being “under”, “below” or “beneath” the second feature may refer to the first feature being directly or obliquely under the second feature or may merely indicate that the horizontal position of the first feature is less than that of the second feature.

It should be noted that, when an element is referred to as “being fixed to” or “being arranged on” another element, the element may be fixed on another element directly or an intermediate element may be present. When an element is considered to be “connected to” another element, the element may be directly connected to another element or an intermediate element may be simultaneously present. The terms “perpendicular”, “horizontal”, “upper”, “lower”, “left”, “right”, and other similar expressions used herein are only for purposes of illustration and are not intended to represent the only implementation.

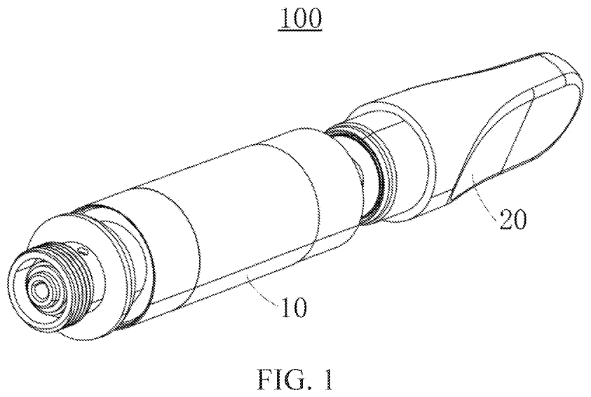

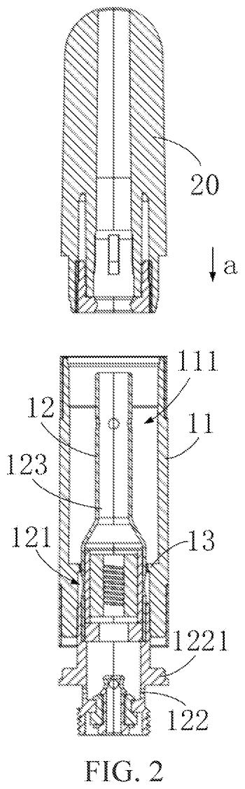

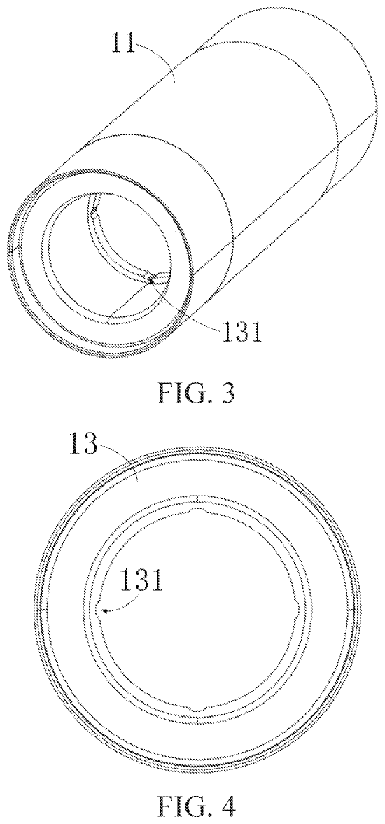

Referring to FIG. 1, FIG. 2, and FIG. 3, an embodiment of the utility model provides a vaporization assembly 10 including a ferrule body 11, a vaporization body 12 and a blocking portion 13. The ferrule body 11 has an inner cavity 111. The vaporization body 12 is adapted into the inner cavity 111 and provided with a liquid inlet hole 121, the blocking portion 13 protrudes from the cavity wall of the inner cavity 111 and is arranged to surround a periphery of the vaporization body 12, and the blocking portion 13 is provided with a liquid-through clearance 131. In addition, the ferrule body 11 can move in a preset direction a relative to the vaporization body 12, and includes a pre-loading position and a conduction position.

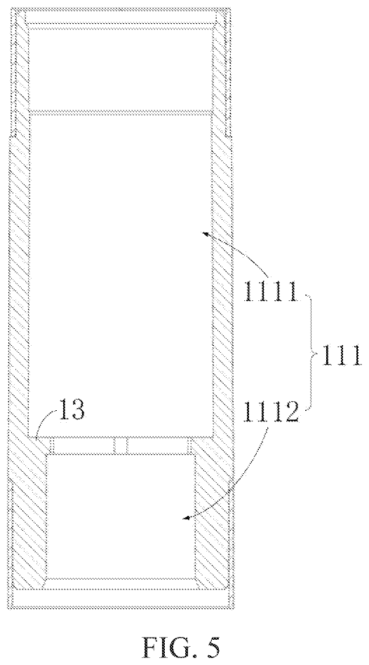

Referring to FIG. 2, FIG. 4, and FIG. 5, when the ferrule body 11 is located at the pre-loading position, the blocking portion 13 partitions the inner cavity 111 into a liquid storage cavity 1111 and a mounting cavity 1112 in the preset direction a, and the liquid inlet hole 121 is located in the mounting cavity 1112 and isolated from the liquid storage cavity 1111.

When the ferrule body 11 moves from the pre-loading position to the conduction position, the volume of the mounting cavity 1112 is reduced, and the liquid in the mounting cavity 1112 is squeezed to flow back into the liquid storage cavity 1111 via the liquid-through clearance 131.

It should be noted that, the vaporization body 12 may heat and vaporize e-liquid. The ferrule body 11 is sleeved outside the vaporization body 12 to protect the vaporization body 12. In addition, the inner cavity 111 of the ferrule body 11 can be used to store e-liquid and assemble the vaporization body 12.

Besides, when the vaporization assembly 10 is in actual use, the liquid injected into the liquid storage cavity 1111 is e-liquid or nicotine liquid, so the liquid in the mounting cavity 1112 is the same as the e-liquid or nicotine liquid in the liquid storage cavity 1111.

Specifically, the blocking portion 13 protrudes from and is arranged to surround the cavity wall of the inner cavity 111. Therefore, the blocking portion 13 partitions the inner cavity 111 into a first sub-cavity and a second sub-cavity. When the vaporization body 12 is adapted into the inner cavity 111, one end of the blocking portion 13 away from the cavity wall of the inner cavity 111 is in tight fit with the vaporization body 12, so that the first sub-cavity and the second sub-cavity are independent of each other. In addition, the ferrule body 11 can move in a preset direction a relative to the vaporization body 12 under the action of an external force. During the relative movement between the ferrule body 11 and the vaporization body 12, the amount of extension of the vaporization body 12 into the first sub-cavity and the second sub-cavity continuously changes.

During the movement of the ferrule body 11 relative to the vaporization body 12, it has a pre-loading position and a conduction position. When the ferrule body is at the pre-loading position, the foregoing first sub-cavity is the liquid storage cavity 1111, and the second sub-cavity is the mounting cavity 1112. Besides, the liquid inlet hole 121 of the vaporization body 12 is located in the mounting cavity 1112, and thus isolated from the liquid storage cavity 1111. In this way, liquid can be injected into the liquid storage cavity 1111. Since the liquid inlet hole 121 is isolated from the liquid storage cavity 1111, the probability that the liquid in the liquid storage cavity 1111 flows into the vaporization body 12 via the liquid inlet hole 121 can be greatly reduced.

However, it can be understood that liquid in the liquid storage cavity 1111 is likely to slowly seep into the mounting cavity 1112 due to the presence of mechanical errors in actual production, such as the fit clearance between the blocking portion 13 and the vaporization body 12, or the structural clearance of the vaporization body 12 itself.

After liquid injection is completed, the ferrule body 11 can move from the pre-loading position to the conduction position under the action of external force, that is, the amount of extension of the vaporization body 12 into the liquid storage cavity 1111 gradually increases. In this way, the vaporization body 12 continues to fill the mounting cavity 1112, causing the volume of the mounting cavity 1112 to gradually reduce. At the same time, the liquid inlet hole 121 follows the vaporization body 12 to move from the mounting cavity 1112 to the liquid storage cavity 1111, and comes into contact with the liquid in the liquid storage cavity 1111.

In the foregoing process, the volume of the mounting cavity 1112 is continuously reduced, which squeezes the liquid in the mounting cavity 1112 to flow back into the liquid storage cavity 1111 via the liquid-through clearance 131 on the blocking portion 13. On the one hand, the liquid can be fully utilized. On the other hand, it may avoid the situation that the liquid in the mounting cavity 1112 is squeezed into the vaporization body 12 during the reduction of the volume of the mounting cavity 1112, and inhaled by the human body during subsequent use.

Further, the blocking portion 13 and the vaporization body 12 may be in an interference fit or a clearance fit. When the blocking portion 13 and the vaporization body 12 are in an interference fit, the airtightness between the blocking portion 13 and the vaporization body 12 can be ensured. However, when the vaporization assembly 10 is in actual use, the liquid injected into the liquid storage cavity 1111 is e-liquid or nicotine liquid, that is, the liquid itself has a higher viscosity. In view of this factor, clearance fit can also be adopted between the blocking portion 13 and the vaporization body 12, and the liquid inlet hole 121 and the liquid storage cavity 1111 can be isolated simply by defining the size of the clearance. For example, the fit clearance between the blocking portion 13 and the vaporization body 12 may be set to 0.01 mm to 0.1 mm. When the fit clearance is within this range, the liquid cannot flow into the mounting cavity 1112 through the clearance due to its own viscosity, thereby also achieving isolation between the liquid inlet hole 121 and the liquid storage cavity 1111.

Besides, in actual use, the way of clearance fit is more beneficial to assembly and therefore, the fitting relationship between the blocking portion 13 and the vaporization body 12 can be set according to the actual circumstance, which will not be repeated herein.

In some embodiments, the liquid-through clearance 131 is arranged at one end of the blocking portion 13 in contact with the vaporization body 12. Therefore, when the ferrule body 11 moves from the pre-loading position to the conduction position, the liquid in the mounting cavity 1112 can flow back into the liquid storage cavity 1111 via the liquid-through clearance 131 along the circumferential wall of the vaporization body 12, so that the liquid in the mounting cavity 1112 can flow back more thoroughly.

In some embodiments, at least two liquid-through clearances 131 are provided and are uniformly arranged in the circumferential direction of the blocking portion 13. The at least two liquid-through clearances 131 arranged uniformly in the circumferential direction of the blocking portion 13 can expand the back flow path and the back flow range for the liquid in the mounting cavity 1112 to flow back into the liquid storage cavity 1111, so that the liquid in the mounting cavity 1112 flows back into the liquid storage cavity 1111 more thoroughly.

Specifically, four liquid-through clearances 131 may be provided and are all uniformly arranged in the circumferential direction of the blocking portion 13. Therefore, the four liquid-through clearances 131 can provide a nearby path for the liquid in the mounting cavity 1112 to flow back, so that the liquid in the mounting cavity 1112 flows back faster. Certainly, in some other embodiments, the quantity of the liquid-through clearances 131 may also be adjusted correspondingly according to actual requirements, which will not be repeated herein.

In some embodiments, the area of the liquid-through clearance 131 ranges from 0.1 square millimeter to 0.5 square millimeter. When the vaporization assembly 10 is in actual use, the e-liquid or nicotine liquid itself has a certain viscosity. Therefore, by controlling the area of the liquid-through clearance 131 to be within a range of 0.1 square millimeter and 0.5 square millimeter, the e-liquid or nicotine liquid in the liquid storage cavity 1111 is less likely to flow into the mounting cavity 1112 via the liquid-through clearance 131 under atmospheric pressure and gravity, but can flow back into the liquid storage cavity 1111 via the liquid-through clearance 131 under the squeezing action caused by the reduced volume of the mounting cavity 1112, thereby achieving a better back flow effect.

In some embodiments, the liquid-through clearance 131 is configured to be a notch formed by recessing an edge of the blocking portion 13 in contact with the vaporization body 12 in the direction away from the vaporization body 12. In this way, the overall structure of the blocking portion 13 is made simpler. In addition, when the vaporization body 12 is adapted into the inner cavity 111, the outer peripheral wall of the vaporization body 12 is in contact with the blocking portion 13. Thereby, the notch recessed on the blocking portion 13 and the outer peripheral wall of the vaporization body 12 can enclose to form a channel for liquid to flow back. When the vaporization body 12 is separated from the inner cavity 111, the recessed notch can facilitate cleaning, thereby avoiding liquid residues in the liquid-through clearance 131.

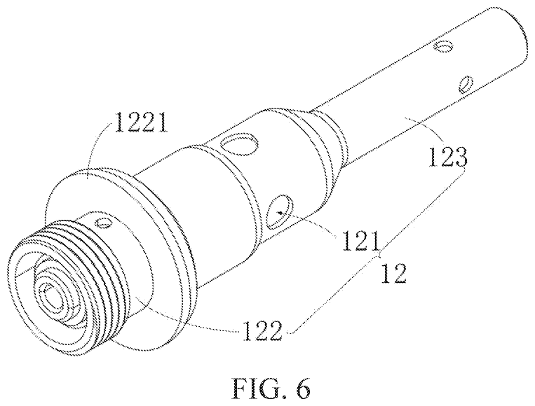

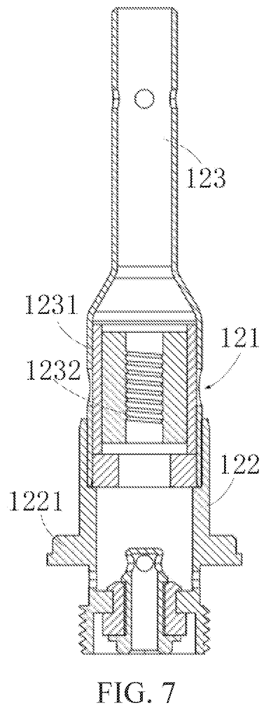

Referring to FIG. 6 and FIG. 7, in some embodiments, the vaporization body 12 includes a vaporization base 122, the surface of the vaporization base 122 protrudes to form a limiting flange 1221, and the ferrule body 11 abuts against the limiting flange 1221 in a limiting manner when moving from the pre-loading position to the conduction position.

Specifically, the vaporization body 12 also includes a vaporization tube 123 which is arranged on the vaporization base 122, and adapted into the inner cavity 111 of the ferrule body 11. The vaporization tube 123 is internally provided with a liquid absorption assembly 1231 and a heating assembly 1232, where the liquid absorption assembly 1231 is arranged corresponding to the liquid inlet hole 121. When the liquid in the liquid storage cavity 1111 enters the vaporization tube 123 via the liquid inlet hole 121, the liquid is absorbed on the liquid absorption assembly 1231, then the liquid absorption assembly 1231 transfers the liquid to the heating assembly 1232, and the heating assembly 1232 heats and vaporizes the liquid so as to facilitate the subsequent inhalation of the vaporized liquid by the human body.

Specifically, the heating assembly 1232 usually includes a ceramic heating body, and the liquid absorption assembly 1231 includes a vaporization cotton sleeved outside the ceramic heating body and arranged corresponding to the liquid inlet hole 121. The vaporization cotton can absorb the liquid in the liquid inlet hole 121 and uniformly transfer the liquid to the ceramic heating body, so that the liquid is more evenly heated by the ceramic heating body.

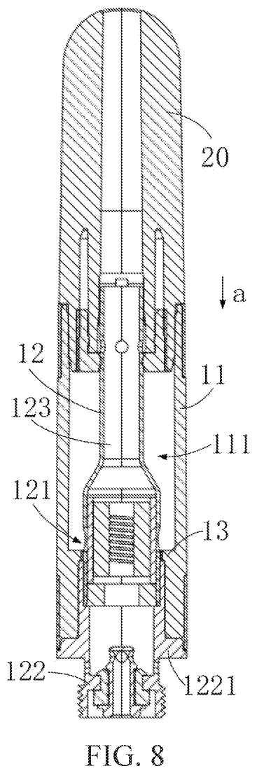

Referring to FIG. 2 and FIG. 8 together, when the ferrule body 11 moves from the pre-loading position to the conduction position, the ferrule body 11 abuts against the limiting flange 1221 in a limiting manner, so that the ferrule body 11 is precisely located at the conduction position through the limiting flange 1221.

In some embodiments, the ferrule body 11 moves between the pre-loading position and the conduction position relative to the vaporization body 12 along its own axis.

Specifically, in this embodiment, the ferrule body 11 is sleeved outside the vaporization body 12 along its own axis, and is coaxial with the vaporization body 12. Thus, the ferrule body 11 can move between the pre-loading position and the conduction position along its own axis, thereby achieving smoother relative movement between the ferrule body 11 and the vaporization body 12.

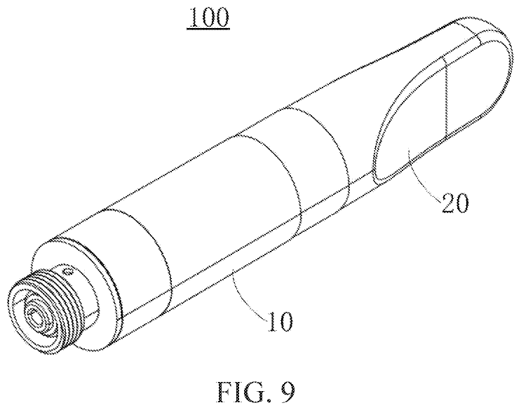

Referring to FIG. 9, based on the same concept as the foregoing vaporization assembly 10, this application provides a vaporizer 100 including a vaporization assembly 10 and a mouthpiece assembly 20. The vaporization assembly 10 is the vaporization assembly 10 as described above, and the mouthpiece assembly 20 is movably adapted onto the ferrule body 11.

Referring again to FIG. 8, specifically, when the mouthpiece assembly 20 is adapted onto the ferrule body 11, the mouthpiece assembly 20 communicates with the vaporization body 12 in the inner cavity 111, and thus can transport the vaporized gas in the vaporization body 12 to the human body, so as to realize the normal use of the vaporizer 100.

In some embodiments, the mouthpiece assembly 20 is separated from the ferrule body 11 when the ferrule body 11 is located at the pre-loading position. The mouthpiece assembly 20, when assembled on the ferrule body 11, presses the ferrule body 11 in the preset direction a to provide a downward pressure for the ferrule body 11 to move from the pre-loading position to the conduction position.

As shown in FIG. 2, specifically, when the ferrule body 11 is located at the pre-loading position, the mouthpiece assembly 20 and the ferrule body 11 are separated from each other. In this way, liquid can be injected into the liquid storage cavity 1111. After liquid injection is completed, the mouthpiece assembly 20 is assembled on the ferrule body 11. During the assembly process of the mouthpiece assembly 20, the mouthpiece assembly 20 presses the ferrule body 11 in the preset direction a to enable the ferrule body 11 to move relative to the vaporization body 12 in the preset direction a. Under the downward pressure of the mouthpiece assembly 20, the ferrule body 11 moves from the pre-loading position to the conduction position, and when the ferrule body 11 is located at the conduction position, the mouthpiece assembly 20 is adapted to the vaporization body 12.

Meanwhile, under the downward pressure of the mouthpiece assembly 20, the volume of the mounting cavity 1112 is continuously reduced, which allows the liquid in the mounting cavity 1112 to flow back into the liquid storage cavity 1111 via the liquid-through clearance 131. As shown in FIG. 8, when the mouthpiece assembly 20 is adapted to the vaporization body 12, the mounting cavity 1112 is filled by the vaporization body 12, and the liquid in the mounting cavity 1112 thoroughly flows back into the liquid storage cavity 1111 via the liquid-through clearance 131.

Based on the same concept as the foregoing vaporizer 100, this application provides an electronic vaporization device including a power supply assembly and the vaporizer 100 described above. The power supply assembly is electrically connected with the vaporizer 100.

In actual use, the ferrule body 11 is located at the pre-loading position in the initial state, and this point, the mouthpiece assembly 20 is separated from the ferrule body 11 and the vaporization body 12. At the same time, the blocking portion 13 partitions the inner cavity 111 of the ferrule body 11 into a liquid storage cavity 1111 and a mounting cavity 1112 in the direction of relative movement between the ferrule body 11 and the vaporization body 12, and the vaporization body 12 successively passes through the mounting cavity 1112 and the liquid storage cavity 1111. The liquid inlet hole 121 in the vaporization body 12 is located in the mounting cavity 1112, and is isolated from the liquid storage cavity 1111.

In this way, liquid can be injected into the liquid storage cavity 1111. After liquid injection is completed, the mouthpiece assembly 20 is assembled on the ferrule body 11, and communicates with the vaporization body 12. During the assembly process of the mouthpiece assembly 20, the mouthpiece assembly 20 presses the ferrule body 11 in the preset direction a to enable the ferrule body 11 to move from the pre-loading position to the conduction position. In the meanwhile, under the downward pressure of the mouthpiece assembly 20, the volume of the mounting cavity 1112 is continuously reduced, which allows the liquid in the mounting cavity 1112 to flow back into the liquid storage cavity 1111 via the liquid-through clearance 131.

When the volume of the mounting cavity 1112 is reduced to zero, the ferrule body 11 abuts against the limiting flange 1221 on the surface of the vaporization base 122 in a limiting manner, so that the ferrule body 11 is located at the conduction position. In this way, the mouthpiece assembly 20 is assembled on the ferrule body 11, and communicates with the vaporization body 12. In addition, the liquid inlet hole 121 is located in the liquid storage cavity 1111, which allows the liquid in the liquid storage cavity 1111 to flow into the vaporization body 12 via the liquid inlet hole 121. The liquid is heated and vaporized in the vaporization body 12, so that vaporized gas obtained after vaporization is conveniently inhaled by the human body via the mouthpiece assembly 20.

The technical features of the above embodiments can be arbitrarily combined. For brevity of description, not all possible combinations of all technical features in the above embodiments are described; however, provided that combinations of the technical features do not conflict with each other, they shall be considered as falling within the scope set forth in the present description.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A vaporization assembly, comprising:
a ferrule body having an inner cavity;
a vaporization body adapted into the inner cavity and provided with a liquid inlet hole; and
a blocking portion protruding from a cavity wall of the inner cavity and arranged to surround a periphery of the vaporization body, the blocking portion being provided with at least one liquid-through clearance,
wherein the ferrule body is movable in a preset direction relative to the vaporization body and comprises a pre-loading position and a conduction position,
wherein, when the ferrule body is located at the pre-loading position, the blocking portion partitions the inner cavity into a liquid storage cavity and a mounting cavity in the preset direction, and the liquid inlet hole is located in the mounting cavity and isolated from the liquid storage cavity, and
wherein, during a movement of the ferrule body from the pre-loading position to the conduction position, a volume of the mounting cavity is reduced so as to squeeze liquid in the mounting cavity to flow back into the liquid storage cavity via the at least one liquid-through clearance.

2. The vaporization assembly of claim 1, wherein the at least one liquid-through clearance is arranged at one end of the blocking portion in contact with the vaporization body.

3. The vaporization assembly of claim 1, wherein the at least one liquid-through clearance comprises at least two liquid-through clearances, the at least two liquid-through clearances being uniformly arranged in a circumferential direction of the blocking portion.

4. The vaporization assembly of claim 1, wherein an area of the at least one liquid-through clearance ranges from 0.1 square millimeter to 0.5 square millimeter.

5. The vaporization assembly of claim 1, wherein the at least one liquid-through clearance comprises a notch formed by recessing an edge of the blocking portion in contact with the vaporization body in a direction away from the vaporization body.

6. The vaporization assembly of claim 1, wherein the vaporization body comprises a vaporization base, a surface of the vaporization base protrudes to form a limiting flange, and the ferrule body abuts against the limiting flange in a limiting manner upon movement from the pre-loading position to the conduction position.

7. The vaporization assembly of claim 1, wherein the ferrule body is configured to move between the pre-loading position and the conduction position relative to the vaporization body along an axis of the ferrule body.

8. A vaporizer, comprising:
the vaporization assembly of claim 1; and
a mouthpiece assembly movably adapted onto the ferrule body.

9. The vaporizer of claim 8, wherein the mouthpiece assembly is separated from the ferrule body when the ferrule body is located at the pre-loading position, and is configured to press the ferrule body in the preset direction when assembled on the ferrule body to provide a downward pressure for the ferrule body to move from the pre-loading position to the conduction position.

10. An electronic vaporization device, comprising:
a power supply assembly; and
the vaporizer of claim 8,
wherein the power supply assembly is electrically connected to the vaporizer.
